(19) United States
(12) Patent Application Publication
(10) Pub. No.: US 2016/0026767 A1
(43) Pub. Date: Jan. 28, 2016

(54) NON-INVASIVE NUTRITION MONITOR

(71) Applicant: THE REGENTS OF THE UNIVERSITY OF CALIFORNIA, Oakland, CA (US)

(72) Inventors: Majid Sarrafzadeh, Anaheim Hills, CA (US); Misagh Falahi, Los Angeles, CA (US); Nabil Alshurafa, Camarillo, CA (US); Suneil Nyamathi, Los Angeles, CA (US); Haik Kalantarian, Los Angeles, CA (US); Adam Ryan Traidman, Mountain View, CA (US)

(73) Assignee: THE REGENTS OF THE UNIVERSITY OF CALIFORNIA, Oakland, CA (US)

(21) Appl. No.: 14/775,586

(22) PCT Filed: Mar. 12, 2014
(86) PCT No.: PCT/US2014/024976

§ 371 Date: Sep. 11, 2015

Related U.S. Application Data
(60) Provisional application No. 61/780,645, filed on Mar. 13, 2013.
(60) Provisional application No. 61/949,179, filed on Mar. 6, 2014.

(51) Int. Cl. G06F 19/00; A61B 7/00; A61B 5/00; A61B 5/11

(52) U.S. Cl. CPC G06F 19/3406; A61B 5/11; A61B 7/008; A61B 5/6822; A61B 5/48

(57) ABSTRACT

An apparatus includes a sensor configured to detect a variable characteristic, the variation of the characteristic including variation indicative of an individual swallowing when the sensor is positioned in a neck area of the individual. The apparatus includes a wireless data communication interface configured to receive information related to the characteristic and transmit the information externally. The sensor may be, for example, an acoustic sensor, a piezoelectric sensor, a capacitive sensor, or a pressure sensor. The apparatus may include a sensor interface to sample a signal from the sensor and provide data related to the signal for transmission externally. A system may use the information related to the characteristic to identify eating habits and type of food eaten. Feedback may be provided to the individual to help the individual change their dietary intake and habits.

### CROSS-REFERENCE TO RELATED PATENT APPLICATIONS

This application claims the benefit of U.S. Provisional Patent Application 61/780,645 filed Mar. 13, 2013 to Falahi et al., titled “NON-INVASIVE NUTRITION MONITOR” and U.S. Provisional Patent Application 61/949,179 filed Mar. 6, 2014 to Sarrafzadeh et al., titled “WEARABLE NUTRITION MONITORING SYSTEM”, the contents of which are incorporated herein by reference in their entirety.

### BACKGROUND

Over-eating may lead to obesity. Obesity is one of the biggest public health issues in many countries around the world. In 2006, the number of overweight people in the world overtook the number of malnourished, underweight people for the first time. In 2008, medical costs associated with obesity were estimated at $147 billion, and as of 2010, 35.7% of American adults were obese. Obesity increases the risk of many negative health consequences, such as coronary heart disease, Type 2 diabetes, high blood pressure, stroke, metabolic syndrome, cancer, osteoarthritis, and hypertension.

Studies have found that eating patterns are associated with being overweight. For example, it has been identified that a greater number of smaller eating episodes each day was associated with a lower risk of obesity. In contrast, skipping breakfast and eating away from home were associated with an increased prevalence of obesity. It has also been found that overweight and obese people are less likely to consume meals regularly, and children from households that regularly eat dinner in front of the television are more likely to eat energy-dense foods such as pizza, snacks and soft drinks, and less likely to eat fruits and vegetables. In addition to eating-related disorders with respect to over-eating, many persons suffer from eating-related disorders with respect to under-eating, such as anorexia and bulimia.

Behavior monitoring may help in the diagnosis and treatment of eating-related disorders. Behavior monitoring includes monitoring of dietary intake.

One technique of monitoring dietary intake is the multipass 24-hour dietary recall, which is based on data individuals provide at the end of a randomly selected day. Each individual gives an oral or written report including the amount and type of dietary intake during the day, as best they recall, which is then used to calculate dietary intake. This approach has significant error because people don't recall the exact amount of dietary intake, and tend to under-report amounts. Experimental data suggests that a minimum number of reports (at least two weekdays and one weekend day) are needed to make a relatively fair judgment using this technique.

Another technique is self-monitoring by way of a food diary, which is similar to 24-hour recall, but individuals record dietary intake preferably directly after eating. However, this requires high adherence, and individuals again tend to under-report dietary intake. There is the additional problem that the act of recording alters the normal choices that people make.

Another technique relies on imaging of food. However, such techniques fail to automatically detect the type of food a person consumes from an image. Such devices also do not inform as to whether individuals actually consume the food captured by the device.

Another technique relies on tracking wrist motion to automatically detect periods of eating. However, tests show relatively low accuracy for detecting eating using this technique as compared to self-monitoring. Further, the technique fails to properly detect habits of people that eat and drink with either hand, and has a high false positive rate (one per five bites) when eating conditions change drastically. Hand gestures will also vary according to social settings and particular gesture habits.

Another technique includes the use of a smart fork that measures eating behavior, including how long it takes to eat and how many bites are taken. However, a major drawback is that individuals have to carry around a special utensil everywhere they go. The technique also fails to detect food consumed by hand such as sandwiches and beverages.

Another technique includes the use of intraoral sensors to identify chewing, which has been shown to be uncomfortable to wear.

Another technique includes the use of a device that fits in the mouth and restricts jaw movement, making an individual take smaller bites, ultimately reducing the amount of food eaten. Such devices create discomfort for the user.

Thus, it would be desirable to have available a non-intrusive automated system for monitoring dietary intake.

### SUMMARY

In one aspect, an apparatus includes a sensor configured to detect a variable characteristic, the variation of the characteristic including variation indicative of an individual swallowing when the sensor is positioned in a neck area of the individual. The apparatus includes a wireless data communication interface configured to receive information related to the characteristic and transmit the information externally. The sensor may be, for example, an acoustic sensor, a piezoelectric sensor, a capacitive sensor or a pressure sensor.

The apparatus may include a sensor interface to sample a signal from the sensor and provide data related to the signal for transmission externally. In one embodiment, the sensor is an acoustic sensor and the characteristic is sound, and the sensor interface includes at least one filter configured to minimize frequencies in the vocal range from the signal. In one embodiment, the sensor is a pressure sensor made from an array of e-textile material, and the signal from the pressure sensor represents changes in resistance of the material. In one embodiment, the sensor is a capacitive sensor made from an array of conductive material, and the signal from the capacitive sensor represents changes in capacitance of the material. In one embodiment, the apparatus may include one or more additional sensors, the sensor interface samples signals from the additional sensor(s), and the transmitted information includes information related to at least two of motion, audible sounds, pressure, bone conductance, and tissue conductance.

In one embodiment, motion information is received via the data communication interface from another device configured to monitor motion of the individual, and wherein the transmitted information includes information related to motion.

In another aspect, a computing device includes a processor-readable medium including processor-executable instructions and a processor configured to execute instructions from the processor-readable medium. The computing device further includes a data communication interface. The processor receives information via the data communication interface, executes a classification process, and identifies from the received information a signal window representing a swallowing motion. In one embodiment, the information received via the data communication interface is acoustic information. The processor may receive motion information via the data communication interface, and analyze the motion information and the acoustic information to determine a health status indicator, and may extract nutritional data from the received information In one embodiment, the processor performs segmentation and feature extraction from the received information.

The processor may communicate with a social networking cite or platform. The processor may estimate dietary intake and provide a visual representation of dietary intake on a display

In one embodiment, the communication interface uses one of Bluetooth, WiFi, XBee, cellular, 3G, and 4G protocols.

In another aspect, a method includes receiving data representative of a signal measured by a sensor positioned adjacent to a throat area of an individual, and segmenting the filtered data into segments of interest. For each segment of interest, the method includes extracting features from the data of the segment, comparing the extracted features with a group of predetermined feature sets, identifying from the comparing a classification of the extracted features, and determining from the classification that the segment does or does not represent a swallowing motion. In one embodiment, the method further includes receiving information representative of a signal measured by a motion sensor positioned on the person or clothing of the individual, and from the information representative of the signal measured by the motion sensor and the data representative of the signal measured by the sensor positioned adjacent to the throat area, determining a health status of the individual. In one embodiment, the method further includes determining, from the filtered data and the classification, a type or category of food eaten by the individual.

### BRIEF DESCRIPTION OF THE DRAWINGS

FIG. 1 illustrates an example of a system for monitoring dietary intake.

FIG. 2 illustrates an example of a computing device.

FIG. 3A illustrates an example of a wearable swallow monitor in the form of a necklace.

FIG. 3B illustrates an example of how the necklace of FIG. 3A may be constructed.

FIG. 4 illustrates an example of a signal from an acoustic sensor, which is filtered to minimize voice and noise information.

FIG. 5 illustrates acoustic signals related to swallowing under different conditions.

FIG. 6 illustrates in overview an example of how a swallow monitoring system may be trained, and used for recognition of swallows.

FIG. 7 illustrates an example of processes for detecting swallows from signals from an audio sensor.

FIGS. 8A-B illustrates a prototype of a wearable swallow monitor.

FIG. 9 illustrates an example of processes for detecting swallows from signals from a piezoelectric sensor.

FIG. 10A illustrates an example of a signal received from a piezoelectric sensor.

FIG. 10B illustrates an example of a smoothed signal.

FIG. 10C illustrates an example of control points identified in a smoothed signal.

FIG. 11A illustrates an example of features detected in a signal.

FIG. 11B illustrates an example of identifying swallows from a signal.

FIG. 12 illustrates an example of processes for detecting swallows from signals from a variety of sensors.

FIG. 13 illustrates an example of a process for classifying swallows.

FIGS. 14A-B illustrate examples of status screens on a graphical user interface.

FIG. 15 illustrates positioning information for piezoelectric sensors used in an experiment.

### DETAILED DESCRIPTION

### Abbreviations

bps: bits per seconds (Mbps: mega bits per second)
dB: decibels (dBA: A-weighted decibels)
g: acceleration due to gravity
Hz: Hertz (kHz: kilohertz)
V: Volts (mV: millivolts)

It is desirable to automate detection of eating habits. A system using automated monitoring may educate an individual on his or her eating patterns and provide suggestions to the individual, such as alternative eating schedules, modified intake amounts, or modified rates of consumption. Providing feedback to individuals about their eating habits via real-time monitoring can help them reach their health and fitness goals, as well as providing guidance with respect to nutritional health, and feedback related to satiation parameters.

Studies have shown that the number of swallows during a day correlate better with weight gain on the following day than did estimates of caloric intake. The system described in this disclosure detects swallowing and categorizes dietary intake based on the swallowing. A mobile wearable device is used to monitor one or more characteristics related to the act of swallowing, such as sound or motion. The monitored characteristic includes information other than swallowing, such as chewing, coughing, sneezing and vocalizing as well as other actions, and may include ambient sound. In some cases, these other motions and sounds may provide information of interest, and in other cases, the information may be filtered out at least partially. From data representing the characteristic(s), the system recognizes swallows, and analyzes dietary intake. Analysis of dietary intake includes, among other analyses, amount and rate of food or liquid ingested, determining a category of food ingested, determining ingestion of medication, and determining eating patterns.

Information regarding dietary intake may be combined with knowledge of physical activity level. The coupling of activity detection and dietary intake detection provides a holistic way to monitor health status and provide suggestions for improvement. Mobile monitoring can help towards a goal of enabling healthier lifestyle choices, and may contribute to behavior modifications. For example, mobile monitoring may allow for treatment of eating-related disorders such as over-eating or under-eating.

In some embodiments of the system described in this disclosure, the system may provide for wireless communication with a mobile device hosting an application (“App”) to allow for: monitoring and feedback while the individual is active; suggestions for times and places to eat; a reminder to wear the monitoring device; feedback on detected eating patterns (normal, over-eating, under-eating), frequency and time of dietary intake for self-modification of behavior; step-by-step guidance to aid in improving eating patterns; and advice on maintaining a balance between activity and nutrition. The App may additionally or alternatively provide other monitoring and feedback capabilities.

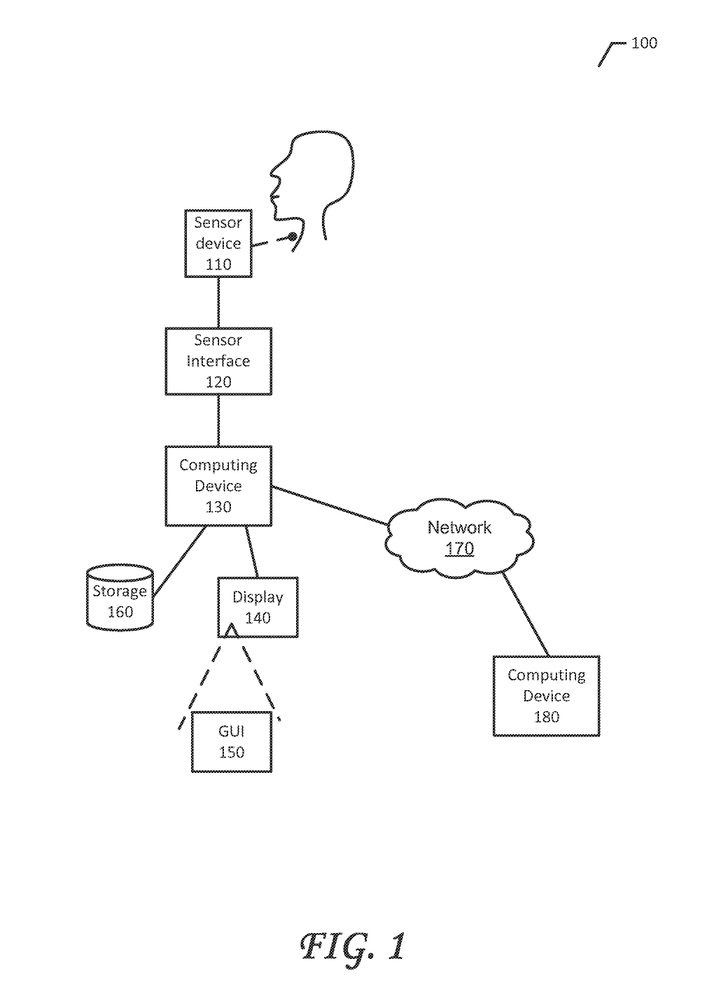

FIG. 1 illustrates a system 100 for automatic monitoring of dietary intake, in which a sensor 110 is positioned along the neck of an individual to detect swallowing. Sensor device 110 is preferably a wearable device, such as a device fashioned in the form of a necklace, scarf, or collar, or embedded in a necklace. Sensor device 110 may be positioned under a patch or secured by an alternate device, or formed as a temporary adhesive device, which may be disposable or reusable. In some implementations, sensor device 110 is an implantable device.

Sensor device 110 may be an auditory sensor, motion sensor, pressure sensor, or other sensor type. Sensor device 110 may represent multiple sensors of the same or different types. Sensor device 110 may output an analog signal, a digital signal, a pulse width modulated signal, or other signal representing the information being sensed.

A sensor interface 120 receives a signal from sensor device 110 and formats the signal for processing. For example, sensor interface 120 may perform one or more of: sample an analog sensor signal to convert the signal to digital form by way of an analog-to-digital converter (ADC); filter a sensor signal or a version of the signal to isolate frequencies of interest and/or to remove noise; convert a digital signal from sensor device 110 or from an ADC to packets of digital information; convert an analog sensor signal to a pulse width modulated signal; convert a pulse width modulated signal to a digital signal or packets of digital information; and normalize a signal. These examples are not limiting. Sensor interface 120 may perform other functions to prepare a signal for processing.

Computing device 130 processes information from sensor interface 120, and may provide a visual representation of the information or an analysis of the information on a display 140 via a graphical user interface (GUI) 150. Computing device 130 may store information from sensor interface 120 and/or data generated from analyses of the information from sensor interface 120 in a storage 160 for later retrieval. Computing device 130 may be, for example, a “smart” phone, a personal digital assistant (PDA), a tablet or other handheld computer, a laptop computer, or a personal computer, or may be a computing portion of another device.

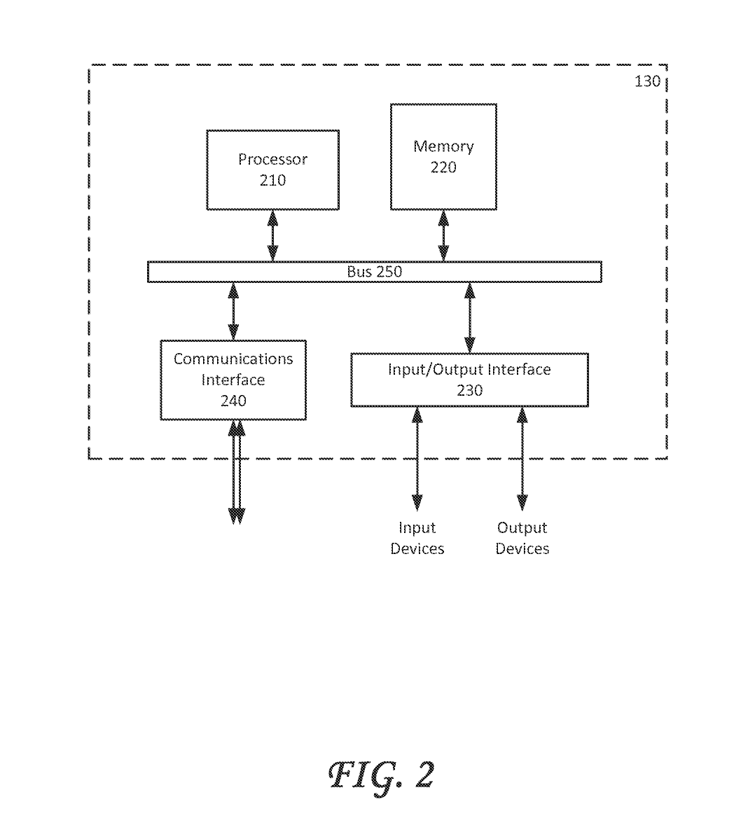

Storage 160 is a memory device, for storing data and instructions. Computing device 130 and storage 160 are described in more detail with respect to FIG. 2.

Computing device 130 may communicate with another computing device 180 over a network 170. For example, computing device 130 may gather and analyze sensor device 110 information from an individual, and provide swallowing information over network 170 to computing device 180 at a physician's office or to a computing device 180 that monitors information about many individuals and stores the information for later retrieval.

The components shown in FIG. 1 are provided by way of illustrating the features of monitoring system 100; however, system 100 may include different components or different arrangements of components. For example: sensor device 110 may be implemented with sensor interface 120; sensor interface 120 may be implemented as part of computing device 130; display 140 and/or storage 160 may be implemented as part of computing device 130; sensor interface 120 may receive information from multiple sensor devices 110; and computing device 130 may receive information from multiple sensor interfaces 120. Communication between various components of FIG. 1 may be via wired or wireless interfaces.

FIG. 2 illustrates an example of a computing device 130 that includes a processor 210, a memory 220, an input/output interface 230, and a communication interface 240. A bus 250 provides a communication path between two or more of the components of computing device 130. The components shown are provided by way of illustration and are not limiting. Computing device 130 may have additional or fewer components, or multiple of the same component.

Processor 210 represents one or more of a processor, microprocessor, microcontroller, ASIC, ASSP, and/or FPGA, along with associated logic.

Memory 220 represents one or both of volatile and non-volatile memory for storing information. Examples of memory include semiconductor memory devices such as EPROM, EEPROM and flash memory devices, magnetic disks such as internal hard disks or removable disks, magneto-optical disks, CD-ROM and DVD-ROM disks, and the like.

Input/output interface 230 represents electrical components and optional code that together provide an interface from the internal components of computing device 130 to external components. Examples include a driver integrated circuit with associated programming.

Communications interface 240 represents electrical components and optional code that together provide an interface from the internal components of computing device 130 to external networks, such as network 170.

Bus 250 represents one or more interfaces between components within computing device 130. For example, bus 250 may include a dedicated connection between processor 210 and memory 220 as well as a shared connection between processor 210 and multiple other components of computing device 130.

Portions of the monitoring system of this disclosure may be implemented as computer-readable instructions in memory 220 of computing device 130, executed by processor 210.

An embodiment of the disclosure relates to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as application-specific integrated circuits (“ASICs”), programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

Computing device 180 may include components similar to the components of computing device 130. Computing device 180 may be any processor-based device, such as a personal computer, server, laptop, handheld computer, or processor-based component of another system. Computing device 180 with network 170 may represent cloud-based computing in some implementations.

In general terms, system 100 includes a mobile wearable sensory device (MWSD) that includes one or more sensor device(s) 110, where sensor data is provided via sensor interface 120 (which may be physically implemented with sensor device 110, or separately implemented) to computing device 130 for analysis, storage, and/or communication. The MWSD includes a wireless transmission unit for transmitting data to computing device 130 and for optionally exchanging information with other devices such as activity monitors. The transmission unit may include, for example, a communication interface such as a wireless Bluetooth, cellular network, RFID, wireless USB, ZigBee, WiFi, 3G, 4G or other wireless interface. Data transmittal may be performed in a secure hardware and/or software environment.

The MWSD includes a battery to allow for mobility. The battery may be rechargeable, such as a rechargeable lithium ion battery. Battery-saving techniques may be implemented in the MWSD for prolonged use.

Data analysis may be performed in the MWSD or computing device 130, or may be partially performed in each of the MWSD and computing device 130. For example, some signal processing and/or analysis may be performed on the MWSD to limit volume of data transmission, for improved MWSD battery life or reduced computing device 130 memory requirements.

Further, in some implementations, computing device 130 receives data from the MWSD, and passes the data to another device (e.g., computing device 180 via network 170) for processing and analysis, and the other device may provide feedback for computing device 130 to present (e.g., at GUI 150). By way of example, data or analysis related to dietary intake (e.g., consumption, rate, type, frequency) may be provided to a remote service (e.g., computing device 180 via network 170), the remote service analyzes the dietary intake information in the context of motion information received from another device (e.g., a Fitbit device), and provides information back to computing device 130 for presentation to the monitored individual. Alternatively in this example, the remote service may provide the motion information received from another device to computing device 130, and analysis of the motion information with the dietary intake information is performed in computing device 130.

Data analysis includes filtering, feature extraction, classification and sensor fusion. Data analysis is used to detect volume and frequency of dietary intake, and thereby determine eating patterns and usage compliance (e.g., that the individual is wearing the MWSD and wearing it properly).

The MWSD may include an activity recognition sensor. The MWSD may communicate with external activity recognition sensors or other devices worn or carried on the person of the individual. Activity recognition sensors include motion detection sensors or systems for calculating energy expenditure. The MWSD may provide information to an activity recognition device, or an activity recognition device may provide information to the MWSD. Analysis of dietary intake and activity information together may allow for improved analysis and correspondingly improved guidance and recommendations, and may further allow the monitoring system to monitor a health status.

Computing device 130 may perform calibration and testing of the MWSDA.

In one embodiment, the MWSD is an MWSD:acoustic (MWSDA) with at least one acoustic sensor such as a microphone. While the MWSDA is active, audio signals from the acoustic sensor are monitored. Time series audio signals at particular frequencies may be used to detect periods of swallowing, and also to monitor usage compliance. Pressure sensors embedded in the MWSDA may further augment audio signals in accurately determining usage compliance in that the distribution of the pressure across the MWSDA may further enhance confidence in a determination that the individual is actually wearing the device.

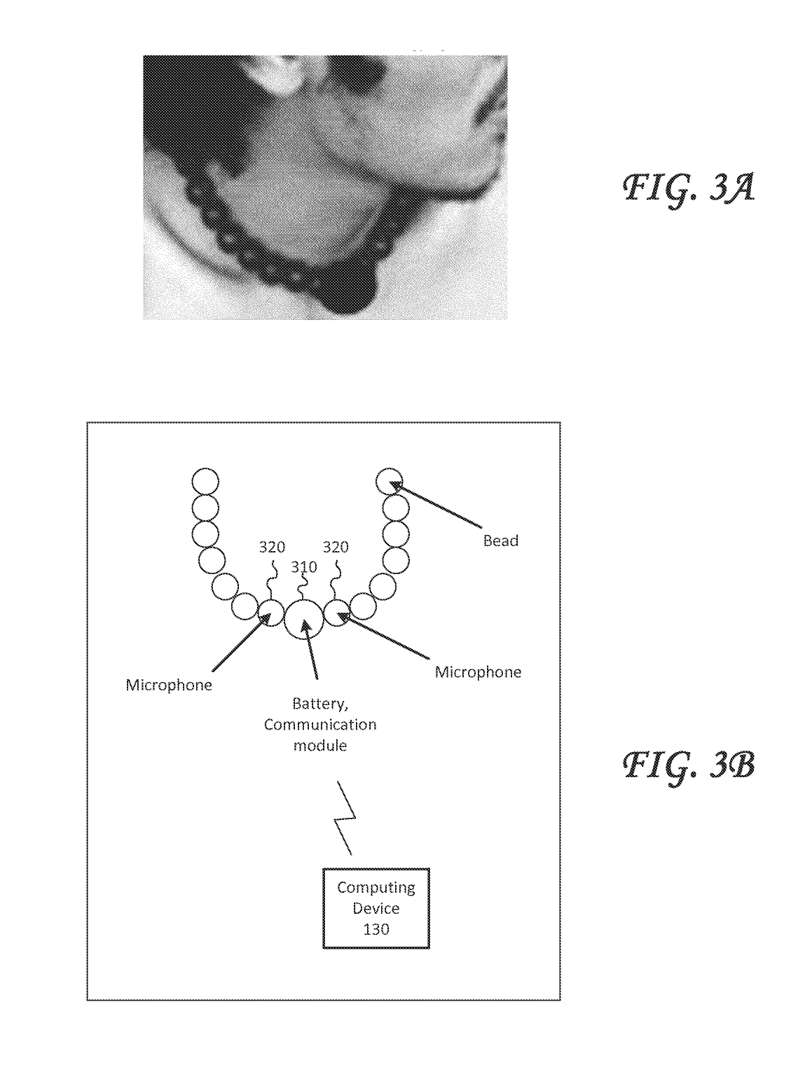

FIG. 3A illustrates a proof of concept prototype of an example of an MWSDA in the form of a necklace, positioned around the neck of a person such that swallowing may be monitored. FIG. 3B illustrates how components of the MWSDA may be discreetly incorporated into the structure of the necklace of FIG. 3A. There are multiple beads extending around the individual's neck in the necklace shown. However, other designs are also possible, such as a necklace including a minimum number of beads to accommodate the components of the MWSDA. In the example shown, the large bead 310 in the center includes a battery and a communications module. The communications module is an FCC certified and fully qualified Bluetooth module with data rates up to 3 Mbps, and includes a low power sleep mode.

The necklace MWSDA of FIGS. 3A-B includes two beads 320, each with a micro electro-mechanical system (MEMS) microphone on a small printed circuit board (PCB). Optionally, one microphone may be used. An amplifier on the PCB has a gain of 67 and produces a peak-to-peak output of about 200 mV for normal conversational volume levels when the microphone is held at arm's length. The signal to noise ratio is −62 dBA, and there is a −3 dB roll off at 100 Hz and again at 15 kHz.

As discussed, embodiments of an MWSD generally may include more than one type of sensor. In the MWSDA example of FIGS. 3A-B, additional sensors may be embedded in the beads, or may be in communication with the necklace via the communication module in bead 310, and information provided to computing device 130 includes information related to the additional sensors. For example, a small, thin, low power, complete triple axis accelerometer with signal conditioned voltage outputs may be added, in which the accelerometer measures acceleration with a minimum full-scale range of +/−3 g, and can measure the static acceleration of gravity in tilt-sensing applications.

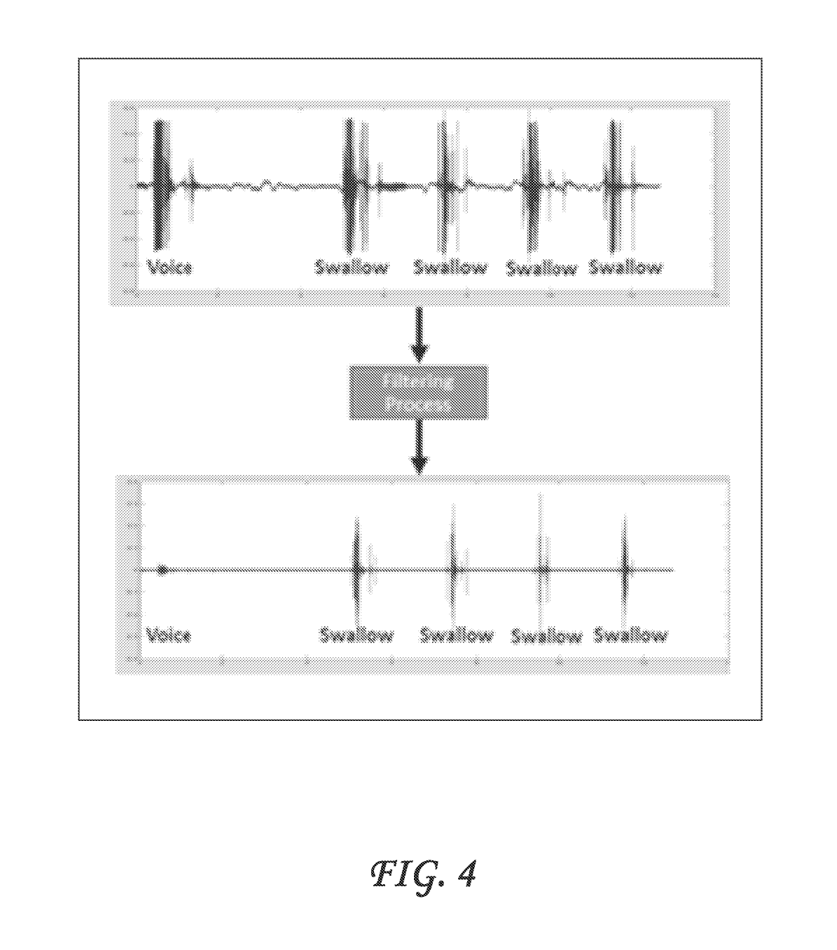

In an MWSDA generally, an audio signal is passed through a set of analog or digital highpass, lowpass, or bandpass filters. The filters are calibrated to diminish signal frequency ranges pertaining to noise and vocal sound. The human voice and swallowing sounds differ by the nature of physical source. The voice is generated by body organs, while swallowing includes the sound of materials making contact. Human voice is concentrated in a range of a few hundred hertz, whereas swallowing sounds exhibit a wider frequency spectrum. Thus, a high-pass filter diminishes most voice sounds while preserving most swallow sounds. In one embodiment, a Chebyshev type II high-pass filter with a cutoff frequency of 4 kHz is implemented. FIG. 4 illustrates that a high-pass filter permits swallowing frequencies to pass while minimizing vocal sounds. Audio spikes generated by inherent properties of a microphone may be filtered out using a low-pass filter with a cutoff frequency of over 10 kHz.

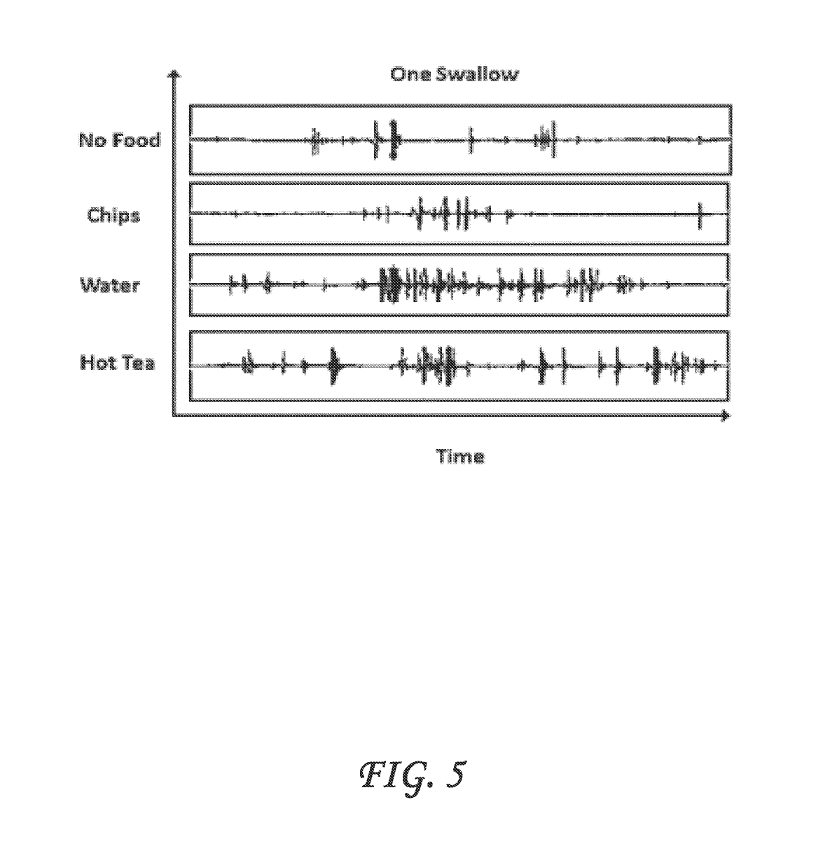

It may be difficult to detect a swallow, because not all swallow motions sound alike. For example, eating solid food may sound very different than drinking a glass of water, and there is even a difference between swallowing a hot or cold drink. FIG. 5 illustrates audio signals for four swallow states by way of example. The state of the swallow is related to the food item and the presence of saliva.

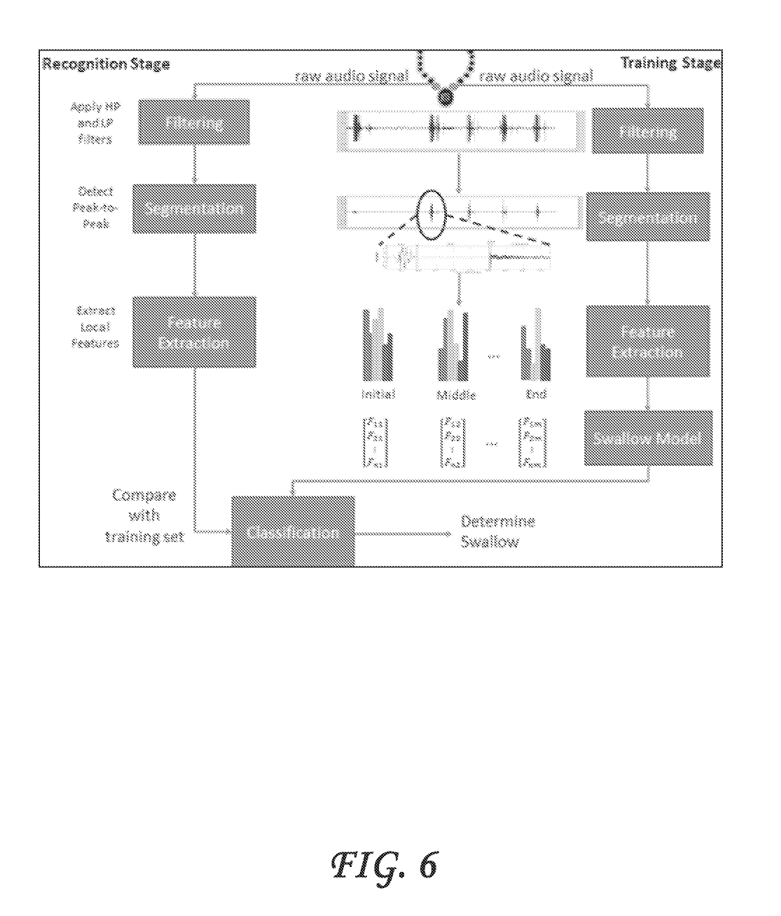

FIG. 6 illustrates an overview of swallow detection using an MWSDA according to this disclosure. The swallow detection includes a training stage and a recognition stage.

In the training stage, swallowing sounds are separated from voice and other sounds using a combination of filtering approaches. After the filtering process, a rolling window averages and normalizes the data, a fast Fourier transform (FFT) at particular frequencies is used to identify several peaks, and the peaks are used for defining segments of the audio data. Each segment of interest may be divided into sub-segments. For example, there may be three sub-segments for a segment of interest, as illustrated by “initial”, “middle” and “end” segments in the example of FIG. 6. A feature matrix may be generated for each segment, or separately for each sub-segment. A swallow model distinguishing swallows from other vocal sounds is created based on feature matrices. Multiple swallow models are generated based on the state of the swallow.

In the recognition stage, audio signals go through a similar process as for the training stage, except that the feature matrix on a new swallow segment is compared against the available swallow models using a classification process. A machine learning process such as nearest neighbor classification, principal component analysis, support vector machine, or the like may be used as a classification process.

Signal features that distinguish between swallows, vocalization and coughs include the number of peaks of a particular length (in seconds), root-mean-square (RMS), waveform fractal dimension (WFD), and power spectrum of the time-domain signal. Power spectrum may be calculated for a segment by applying a Hanning window, using an FFT on the windowed segment, and averaging over different frequency bands from 50 Hz to 1500 Hz, for example. Mean power frequency and peak frequency may be calculated from the power spectrum of each segment.

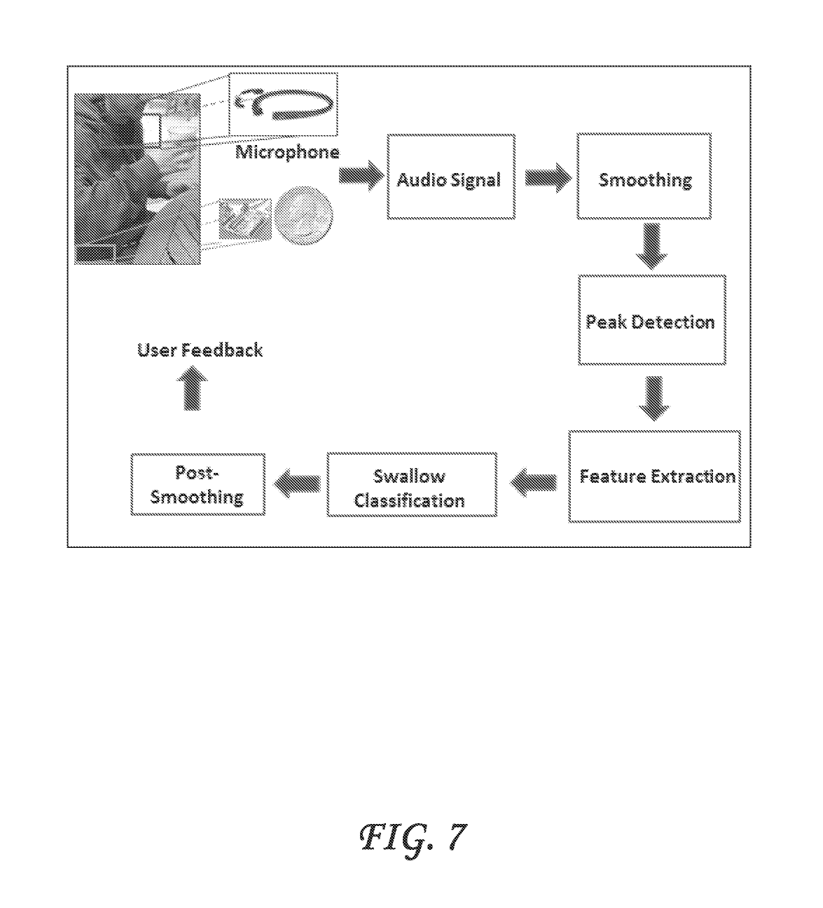

FIG. 7 illustrates visually an example of signal processing that may be performed in a system including an MWSDA. The audio signal from an auditory sensor (e.g., microphone) is smoothed, and peaks are detected in the signal. Features are extracted from the signal, and the features used to classify sounds as representing swallowing. Post-process smoothing minimizes incorrect detection of swallows. Information related to the swallows is provided as user feedback.

In another embodiment, the MWSD is an MWSD:piezoelectric (MWSDP) with at least one piezoelectric sensor. Electric charge is generated on a piezoelectric material when subjected to mechanical stress. Thus, the piezoelectric sensor of the MWSDP deforms during each swallow event due to motion in the throat, and the resulting voltage change at the terminals of the piezoelectric sensor is sampled. An MWSDP may include an array of piezoelectric sensors to provide a larger area of detection around the neck, thus making the MWSDP easier to position while also enhancing the detection and potential classification of swallow motions.

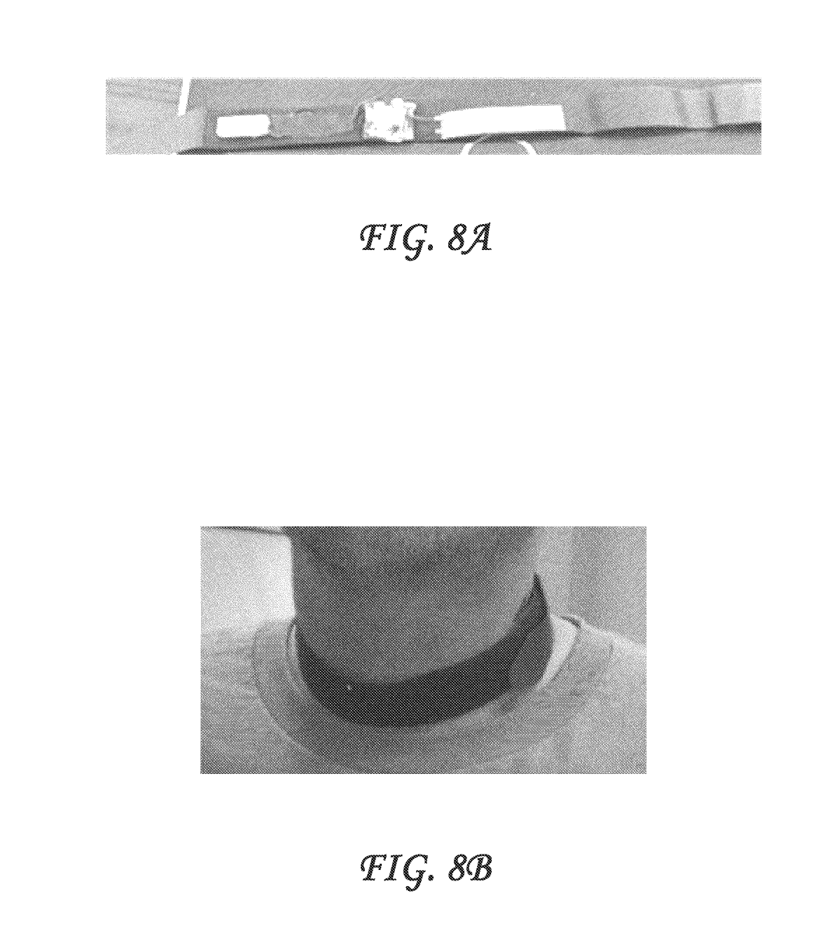

FIG. 8A illustrates a proof of concept prototype for an embodiment of an MWSDP, positioned in the throat area as illustrated in FIG. 8B such that the piezoelectric sensor contacts the throat area. The prototype MWSDP includes a wearable band to which a piezoelectric sensor is attached, and a unit attached to the piezoelectric sensor including a microprocessor, Bluetooth module and battery (the unit may be, for example, attached to the wearable band or attached to clothing, such as attached to a belt). In this prototype, the unit is an Arduino-compatible board that communicates externally using a Bluetooth 4.0 LE transceiver based on an RFD22301 SMT module. The Bluetooth module is fully FCC certified, with data rates up to 3 Mbps and a low power sleep mode. The processor in the unit is an ARM Cortex M0 with 256 kilobytes (kB) of flash memory and 16 kB of RAM. The battery is a small coin 3.3 V rechargeable Lithium ion cell battery.

The prototype MWSDP has an associated application (App) for a smart phone, which communicates with the MWSDP via Bluetooth. The App processes the sensor data and provides feedback to the user, including showing the number of swallows accrued in real time throughout the day. Processing of the data includes smoothing the signal to emphasis the information of interest while removing noise and other information in the data. Peaks and valleys (referred to as control points) are detected in the data, identifying motions in the esophagus which potentially indicate a swallowing motion. Several features are extracted from the signal data for a time before, during, and after the control points. The features are then compared to a predetermined classification scheme to identify which control points represent swallowing motions. A post-processing filter is applied to identify incorrect classifications, such as identified swallow sequences that would not actually occur.

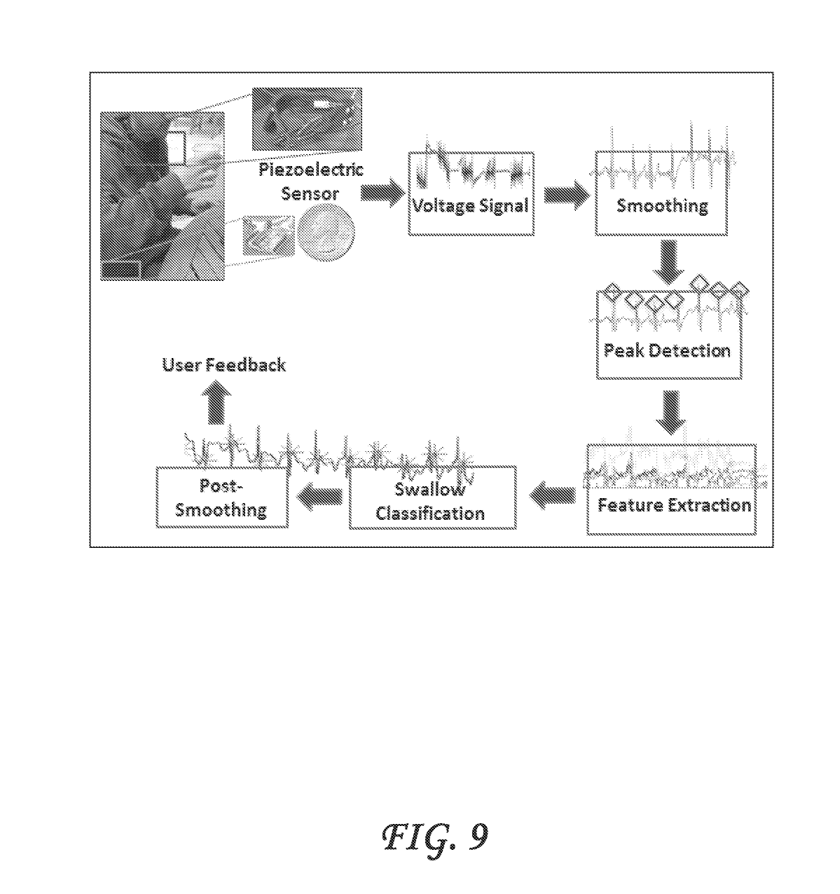

FIG. 9 illustrates visually an example of signal processing that may be performed in a system including an MWSDP. The signal from the piezoelectric sensor is smoothed, and peaks are detected in the signal. Features are extracted from the signal, and the features used to classify signal information as representing swallowing. Post-process smoothing minimizes incorrect detection of swallows. Information related to the swallows is provided as user feedback.

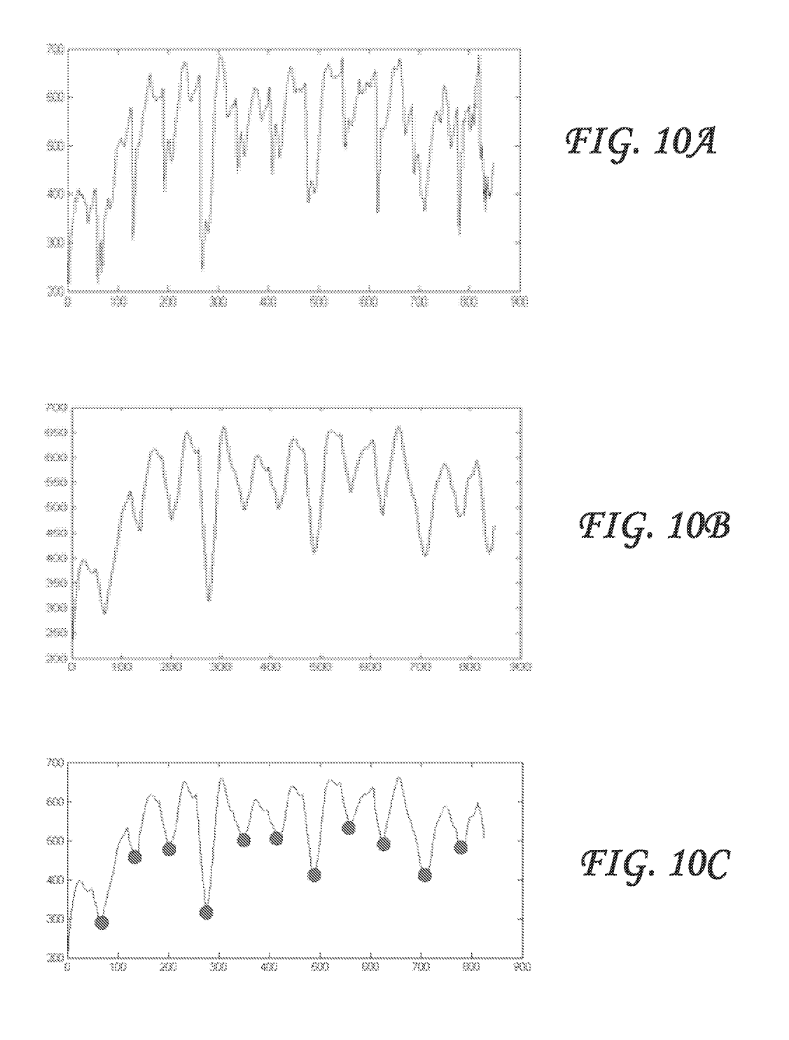

FIG. 10A illustrates by way of example a signal representing data received from the piezoelectric sensor. FIG. 10B illustrates the signal after a smoothing filter is applied. FIG. 10C illustrates control points identified at the valleys of the smoothed signal.

Peaks and valleys of the voltage signal may indicate swallowing motion, but also may indicate chewing, motion of the individual, or noise in the signal. Therefore, after identifying control points (peaks and valleys), signal features around the control points are extracted.

Signal feature include mean, standard deviation, and energy of the signal, and correlation between portions of the signal. The mean of the voltage signal calculated over the feature extractor window is the DC component of the signal, which is useful in capturing the range of possible swallows that may look similar in nature but differ in speed of swallow. The energy of the signal, obtained either in the time or frequency domain, is a measurement of the intensity of a swallow. Other features may also be extracted. In the prototype, 45 features were identified per segment. The features included mean, median, minimum, maximum, standard deviation, energy, interquartile range, skewness, zero crossing rate, variance, mean crossing rate, kurtosis, first derivative, second derivative, and third derivative.

Extracted features are applied to a decision tree to determine which of the control points represent swallows. The decision tree used was developed along with the prototype, and outperforms other techniques such as as SVM, kNN, Bayesian Networks and C4.5 Decision Trees in classifying swallows.

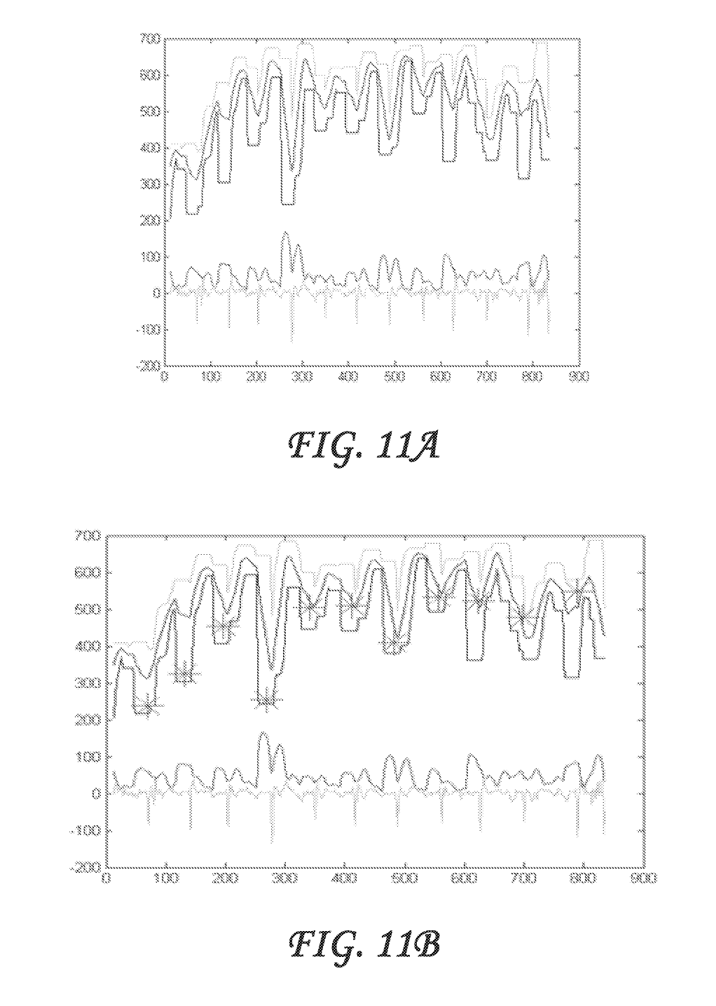

FIG. 11A illustrates graphically some of the features identified in the filtered signal shown in FIG. 10B. FIG. 11B illustrates swallows classified from the features, where each swallow classified is denoted by a star.

Two embodiments of an MWSD (an MWSDA and an MWSDP) have thus been described. Other embodiments of an MWSD include the use of any combination of auditory sensor, vibration sensor, pressure sensor, resistive sensor, and capacitive sensor. Pressure sensors, in one implementation, are made from an array of e-textile material, which detect changes in resistance of the material due to pressure applied to the material (e.g., from swallowing). Capacitive sensors, in one implementation, are made from an array of conductive material, which detects changes in capacitance due to pressure applied to the material (e.g., from swallowing). Data from multiple sensors may be fused by the processor.

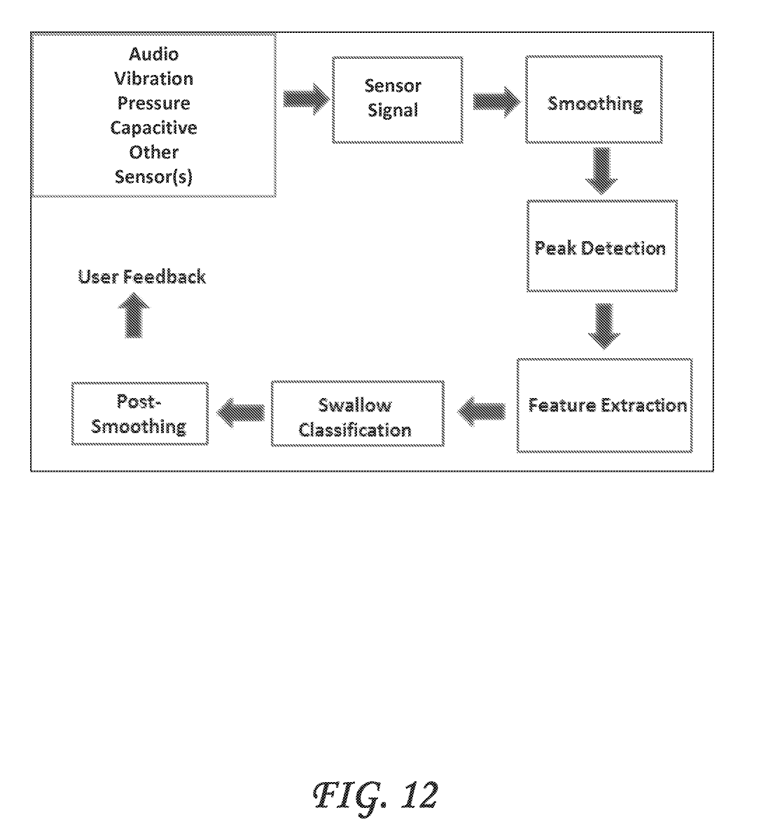

FIG. 12 illustrates generally the MWSD system described in this disclosure. A sensor, or two or more sensors in any combination, are used to detect a characteristic of the sensor environment such as sound, motion, pressure, or capacitance. Signals from the sensor(s) are applied to a smoothing filter, and control points of the signals identified. Features around the control points are extracted and used to classify the control points as indicating swallows or not indicating swallows. A post-processing smoothing filter is used to remove classifications that are probably not true swallows. Feedback may be provided to a user.

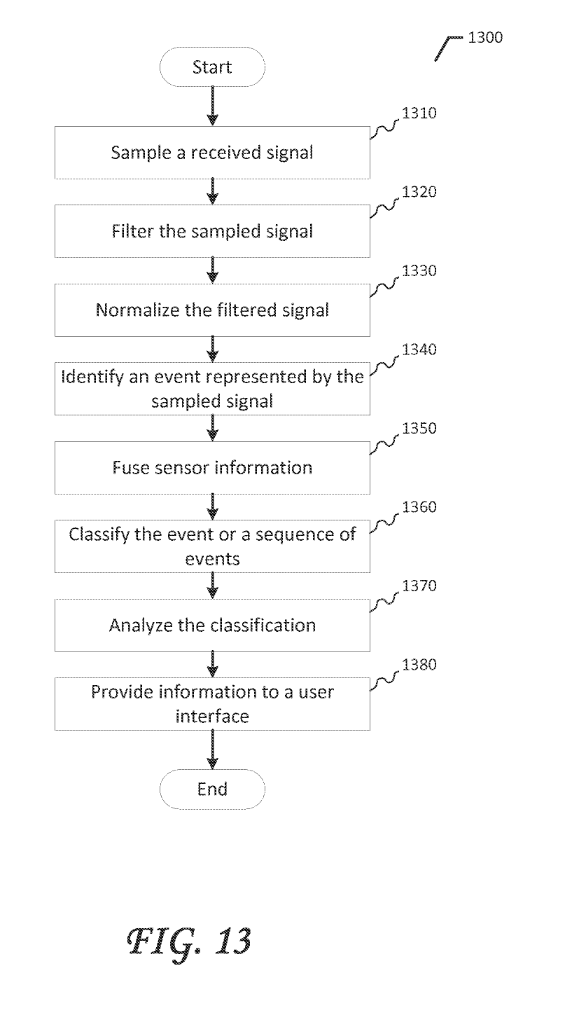

FIG. 13 illustrates an example of a process 1300 for monitoring dietary intake. Process 1300 starts at block 1310, in which a signal received from a sensor device 110 is sampled by sensor interface 120. The sampled signal is filtered (block 1320) and normalized (block 1330) by sensor interface 120 or computing device 130. An event is identified from the normalized signal (block 1340) by computing device 130. An event may be a peak or valley, or a window including one or more peaks or valleys, or a window including one or more features of interest. When signals are received from multiple sensor devices 110, information from the multiple sensor devices 110 may be fused (block 1350). At block 1360, an event (or a sequence of events) is classified as representing a swallowing motion. At block 1370, the classification of a swallow motion is used in the analysis of dietary intake. At block 1380, information regarding dietary intake, swallows, health status, or the like is provided as user feedback. Process 1300 ends after block 1380.

As can be seen below with respect to Experimental Results, information related to swallows may be used to classify dietary intake into categories. Signal events identified as not related to swallowing may also provide useful information, such as classifications of sneezing or coughing that indicate an onset, progression, or status of an illness; or classifications of idle time that indicate excessive times of inactivity.

Additionally, the classifications of motions provide the capability of predicting when a swallow is about to occur, and what will be swallowed (e.g., a category of food or liquid, a medication, or a swallow with no dietary or medicine intake).

Generally, an MWSD App executing on a computing device 130 receives information, runs filters, classifies the data and detects dietary intake. The App can also distinguish between solid food, liquid, talking, and idle time. The App may run in the background to continuously monitor swallowing activity. Signal data and statistics calculated by the App may be displayed, for example on GUI 150 of display 140. Statistics may include feature statistics, or statistics related to dietary intake and activity. For example, statistics may include the fraction of time spent in each of various activities, fractions of food types ingested in a time period, rate of eating, amount of hydration in a time period, amount of dietary intake in a time period (e.g., estimated volume of food during the present meal or daily total), and so forth. The App may alert the user if a high rate of swallows is detected within a particular category over a given time period, or if unusual eating habits are detected, such as cases in which a meal is found to be substantially larger than the recent average for that time of day. Excessive snacking, skipping meals, inadequate hydration levels, and time in which the MWSD is removed may also be reported. The App is also able to perform a classification of food types into categories, helping users to incorporate sound nutritional balance in their diet. The App may allow a user to view results, store them, and set specific time frames to record data. The App may automatically store statistics and alerts, for later retrieval by a third party (e.g., a physician), and some portions of the App may be password locked so that, for example, automatic storage of data may not be disabled. In some implementations, the App provides information remotely through a communications interface on the host computing device, and the information may be provided on a schedule, at the occurrence of an event, or on request. The GUI may provide advice to the user, and connect the user to a social network of users to help create a strong nutrition and health support group.

User feedback includes, for example, text at GUI 150, vibration (e.g., of a smart phone), visual cues (e.g., a flashing LED), or as audio playback via an embedded speaker. Feedback regarding an individual's eating habits may be provided, and the feedback may be based on short-term monitoring or long-term monitoring. For example, feedback may be provided at the granularity of a single meal, as well as being provided as long-term trends in dietary habits. Statistics about the individual's dietary intake and trends or changes in dietary intake may be uploaded to a secure website for long-term tracking and analytics.

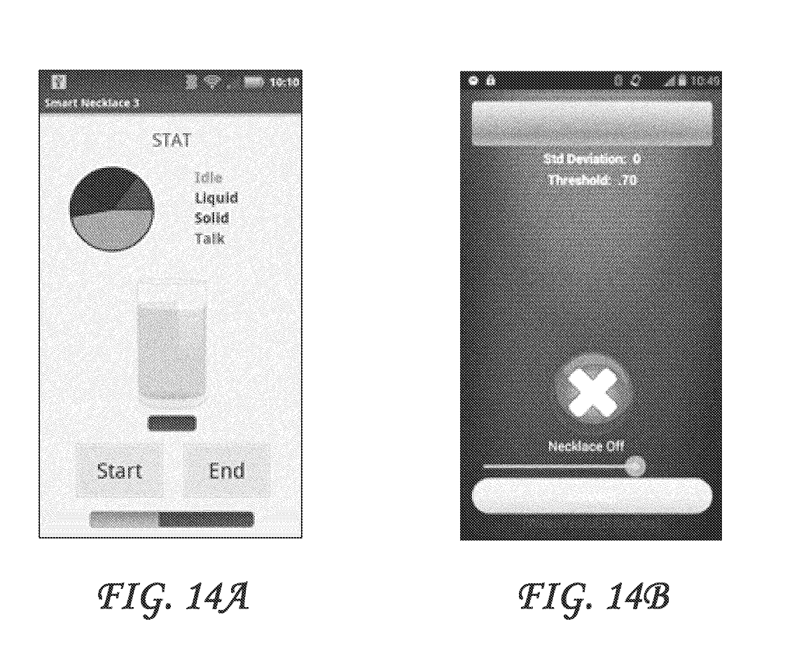

FIG. 14A illustrates one example of a status screen in a smartphone application. FIG. 14B illustrates another example of a status screen, indicating that the MWSD (e.g., “necklace”) has been removed. Other screens may include, for example, a graph that visually shows eating patterns throughout the day, where the graph may be “zoomed” in and out to see swallow distribution throughout the day.

### Experimental Results

### MWSDA

Using a prototype MWSDA and prototype analysis processes, audio data was recorded for five subjects for one hour, as summarized in Table 1. Each of the subjects were recorded in seven states: eating nothing, eating chips, eating cookies, eating Mentos candy, chewing gum, drinking cold water, and drinking hot tea. The swallowing rate (swallows per minute) was measured for various subjects and types of chewing (“none” indicates no chewing activity).

TABLE 1
Variation in swallow count per food type

| | Food Type | | | | | | |
|---|---|---|---|---|---|---|---|
| Subject | None | Chips | Cookies | Mentos | Gum | Water | Hot Tea |
| 1 | 2 | 4 | 4 | 5 | 4 | 16 | 8 |
| 2 | 2 | 3 | 5 | 8 | 7 | 42 | 18 |
| 3 | 6 | 3 | 4 | 5 | 7 | 16 | 14 |
| 4 | 2 | 5 | 5 | 5 | 6 | 24 | 8 |
| 5 | 4 | 4 | 2 | 5 | 3 | 34 | 13 |
| Average | 3.2 | 3.8 | 4 | 5.6 | 5.4 | 26.4 | 12.2 |

Table 1 suggests that there is a relationship between swallow rate and type of dietary intake.

Table 2 provides swallow detection accuracy of the prototype MWSDA system for the experiment outlined with respect to Table 1.

TABLE 2
Accuracy of prototype in detecting swallows

| | Food Type | | | | | | |
|---|---|---|---|---|---|---|---|
| Subject | None | Chips | Cookies | Mentos | Gum | Water | Hot Tea |
| 1 | 89% | 95% | 94% | 90% | 75% | 70% | 84% |
| 2 | 91% | 90% | 95% | 85% | 93% | 80% | 86% |
| 3 | 88% | 93% | 90% | 86% | 89% | 84% | 90% |
| 4 | 80% | 85% | 86% | 80% | 90% | 85% | 95% |
| 5 | 85% | 86% | 89% | 79% | 82% | 85% | 83% |
| Average | 87% | 90% | 91% | 84% | 86% | 81% | 88% |

### Experimental Results

### MWSDP

Using a prototype MWSDP and prototype analysis processes, piezoelectric data was recorded for ten subjects for one hour, as summarized in Table 3. Each of the subjects were recorded in four states: eating a 3-inch sub sandwich, eating a 6-inch sub sandwich, drinking an 8 ounce (oz.) glass of water, and drinking an 18 oz. glass of water. The number of swallows was measured. As can be seen from the results, food and drink portions may be distinguished based on the number of swallows.

TABLE 3

| | | Sandwich | Sandwich | Water | Water |
|---|---|---|---|---|---|
| Subject | Gender | (3-inch) | (6-inch) | (9 oz) | (18 oz) |
| 1 | Male | 11 | 19 | 8 | 13 |
| 2 | Male | 9 | 21 | 7 | 15 |
| 3 | Male | 9 | 25 | 11 | 21 |
| 4 | Male | 25 | 48 | 8 | 12 |
| 5 | Female | 15 | 38 | 17 | 45 |
| 6 | Male | 13 | 29 | 12 | 19 |
| 7 | Male | 9 | 32 | 9 | 18 |
| 8 | Female | 22 | 41 | 13 | 33 |
| 9 | Male | 15 | 30 | 21 | 38 |
| 10 | Male | 8 | 23 | 9 | 23 |
| | Average | 13.6 | 30.6 | 11.5 | 23.7 |

Positioning of piezoelectric sensors was studied in another test. For each of ten subjects, six locations on the neck were tested. The sensors were placed snug against the skin, but not so tight as to be uncomfortable.

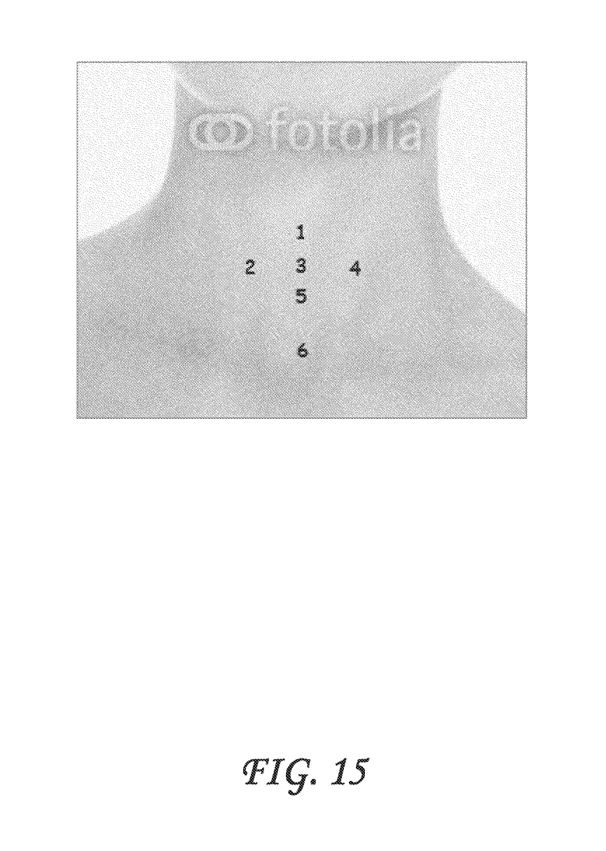

FIG. 15 illustrates the six tested positions, described as follows:

Position 1: a bit below the Adam's apple and approximately 1 cm above position 3

Positions 2 and 4: approximately 1 cm to the left and right, respectively, of position 3

Position 3: approximately 1 cm above position 5

Position 5: at the lowest part of the throat, with the sensor horizontally centered

Position 6: approximately 1 cm below position 5, not on the throat

Each of the subjects were recorded in four states: drinking a 6 oz. cup of room temperature water; eating 5 plain Pringles potato chips one at a time; eating a small sandwich (approximately five bites) made with ground meat, cheese, and lettuce. Portions were measured so as to substantially be the same for each subject. Test results overall are shown in Table 4. Test results for each position are shown in Tables 5-10. As can be seen from Tables 4-10, consistent results were achieved across all positions 1-6.

TABLE 4

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

TABLE 5
Position 1

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

TABLE 6
Position 2

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

TABLE 7
Position 3

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

TABLE 8
Position 4

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

TABLE 9
Position 5

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

TABLE 10
Position 6

| | Food Type | | |
|---|---|---|---|
| Subject | Water | Sandwich | Chips |
| 1 | 89% | 95% | 94% |
| 2 | 91% | 90% | 95% |
| 3 | 88% | 93% | 90% |
| 4 | 80% | 85% | 86% |
| 5 | 85% | 86% | 89% |
| 6 | 89% | 95% | 94% |
| 7 | 91% | 90% | 95% |
| 8 | 88% | 93% | 90% |
| 9 | 80% | 85% | 86% |
| 10 | 85% | 86% | 89% |
| Average | 87% | 90% | 91% |

In the prototype systems described, the majority of the signal processing takes place on the computing device in terms of detecting the swallows. In other embodiments, processing may be performed within the MWSD.

In sum, the system described in this disclosure assesses when individuals consume food and what types of foods were consumed. Different sensors may be used to be able to monitor different food categories and types. The system can help individuals towards goals of weight loss/gain, weight maintenance, correcting bad eating patterns, or improved nutrition. The system is easy to use, detects good and bad eating patterns, and is relatively inexpensive compared to other techniques. The system may be combined with physical activity monitors to provide feedback on both nutrition and activity, helping an individual lead a more balanced lifestyle. The system could be used to diagnose and/or treat disorders such as dysphagia.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

What is claimed is:

1. A wearable apparatus, comprising:
a sensor configured to detect a variation of a characteristic, the variation of the characteristic indicative of an individual swallowing when the sensor is positioned in a neck area of the individual;
an affixing member coupled to the sensor, the affixing member configured to engage a body part of the individual and to position the sensor in the neck area of the individual; and
a wireless data communication interface coupled to the sensor and configured to transmit information related to the characteristic externally.

2. The apparatus of claim 1, wherein the sensor is a piezoelectric sensor.

3. The apparatus of claim 1, wherein the sensor is a pressure sensor.

4. The apparatus of claim 1, further comprising a sensor interface configured to sample a signal from the sensor and provide data related to the signal for transmission externally.

5. The apparatus of claim 4, wherein the sensor is an acoustic sensor and the characteristic is sound.

6. The apparatus of claim 5, wherein the sensor interface includes at least one filter configured to attenuate frequencies in the vocal range from the signal.

7. The apparatus of claim 4, further comprising at least one additional sensor, wherein the sensor interface is further configured to sample signals from the at least one additional sensor, and the transmitted information includes information related to at least two of motion, audible sounds, and pressure.

8. The apparatus of claim 1, wherein motion information is received via the data communication interface from another device configured to monitor motion of the individual, and wherein the transmitted information includes information related to motion.

9. A computing device, comprising:
a processor-readable medium including processor-executable instructions;
a processor configured to execute instructions from the processor-readable medium; and
a data communication interface;
wherein the processor-executable instructions include instructions to receive information via the data communication interface, execute a classification process on the received information, and identify from the received information a signal window representing a swallowing motion.

10. The computing device of claim 9, wherein the information received via the data communication interface is acoustic information.

11. The computing device of claim 10, wherein the processor-executable instructions further include instructions to receive motion information via the data communication interface, and analyze the motion information and the acoustic information to determine a health status indicator.

12. The computing device of claim 9, wherein the processor-executable instructions further include instructions to extract nutritional data from the received information.

13. The computing device of claim 9, wherein the processor-executable instructions further include instructions to perform segmentation and feature extraction from the received information.

14. The computing device of claim 9, wherein the processor-executable instructions further include instructions to communicate with a social networking cite or platform.

15. The computing device of claim 9, wherein the processor-executable instructions further include instructions to estimate dietary intake and provide a visual representation of dietary intake on a display.

16. The computing device of claim 9, wherein the communication interface is configured to transmit information according to at least one of Bluetooth, WiFi, XBee, cellular, 3G, and 4G protocols.

17. A method comprising:
receiving data representative of a signal measured by a sensor positioned adjacent to a throat area of an individual;
segmenting the data into segments of interest; and
for each segment of interest:
extracting features from the data of the segment;
comparing the extracted features with a group of predetermined feature sets;
identifying from the comparing a classification of the extracted features; and
determining from the classification that the segment represents a swallowing motion.

18. The method of claim 17, further comprising:
receiving data representative of a signal measured by a motion sensor positioned on the individual or clothing of the individual; and
from the data representative of the signal measured by the motion sensor and the data representative of the signal measured by the sensor positioned adjacent to the throat area, determining a health status of the individual.
